(19) United States
(12) Patent Application Publication
(10) Pub. No.: US 2026/0341499 A1
(43) Pub. Date: Oct. 29, 2026

(54) COMMUNICATION INTERFACE FOR MEDICAL DEVICES

(71) Applicant: ALCON INC., Fribourg (CH)

(72) Inventors: Aakash NAGARAJAN, Arlington, TX (US); Sinchan BHATTACHARYA, Fort Worth, TX (US); Ramesh SARANGAPANI, Coppell, TX (US); Nikhil SCARIA, Fort Worth, TX (US); Vignesh SURESH, Fort Worth, TX (US); Zhuoran WU, Rancho Cucamonga, CA (US); Lu YIN, Fort Worth, TX (US)

(21) Appl. No.: 19/645,084

(22) Filed: Apr. 10, 2026

Related U.S. Application Data
(60) Provisional application No. 63/794,891, filed on Apr. 25, 2025.

(51) Int. Cl. G16H 40/67

(52) U.S. Cl. CPC G16H 40/67

(57) ABSTRACT

A method of interfacing with medical devices may include obtaining, at a communication interface, image data from a medical device and obtaining, at the communication interface, presentation data generated by an inference system configured to perform artificial-intelligence inference on the image data. The method may further include mapping the presentation data to a data structure accepted by the medical device, the data structure configured to direct the medical device how to render the presentation data; and directing the data structure to the medical device for processing by the medical device.

### CROSS-REFERENCE TO RELATED APPLICATION

This application claims priority to U.S. Provisional Application No. 63/794, 891, filed on Apr. 25, 2025, which is incorporated herein in its entirety.

### FIELD

The embodiments discussed herein relate to a communication interface for medical devices.

### BACKGROUND

Medical imaging systems in surgical environments generate continuous streams of visual data during procedures. Surgical devices such as microscopes, endoscopes, and visualization consoles capture real-time video of the surgical field. Operating room equipment from different manufacturers employs proprietary communication protocols and data formats for transmitting information between devices. Surgical visualization systems typically display basic instrument parameters such as pressure readings, flow rates, and device status information overlaid on live video feeds. These overlay capabilities utilize existing rendering layers within the display devices to present supplementary information to surgeons during procedures. Current surgical devices possess limited onboard computational resources, restricting their ability to perform complex data processing tasks locally.

The subject matter claimed herein is not limited to embodiments that solve any disadvantages or that operate only in environments such as those described above. Rather, this background is only provided to illustrate one example technology area where some embodiments described herein may be practiced.

### SUMMARY

According to an aspect of at least one embodiment, a method of interfacing with medical devices includes obtaining, at a communication interface, image data from a medical device and obtaining, at the communication interface, presentation data generated by an inference system configured to perform artificial-intelligence inference on the image data. The method may further include mapping the presentation data to a data structure accepted by the medical device, the data structure configured to direct the medical device how to render the presentation data; and directing the data structure to the medical device for processing by the medical device.

The object and advantages of the embodiments will be realized and achieved at least by the elements, features, and combinations particularly pointed out in the claims. It is to be understood that both the foregoing general description and the following detailed description are explanatory and are not restrictive of the invention, as claimed.

### BRIEF DESCRIPTION OF THE DRAWINGS

Example embodiments will be described and explained with additional specificity and detail through the use of the accompanying drawings in which:

FIG. 1 illustrates a block diagram of an environment for interfacing local computing devices with medical devices;

FIG. 2 illustrates a flowchart of an example method of interfacing with medical devices;

FIG. 3 is a block diagram of an example computing system for generative artificial intelligence; and

FIG. 4 is a block diagram of an example computing device, all in accordance with at least some embodiments of the present disclosure.

### DETAILED DESCRIPTION

Modern surgical procedures, particularly ophthalmic surgeries, increasingly rely on advanced visualization systems to guide surgeons during complex operations. Surgical display devices present live video streams of the surgical field, often augmented with basic information such as device parameters, pressure readings, or intraocular coherence values. However, existing surgical visualization systems face significant limitations when attempting to integrate sophisticated artificial intelligence-generated information into the surgical workflow.

One challenge arises from the computational constraints of surgical display devices. Many surgical devices lack sufficient onboard processing capability to execute complex AI (Artificial Intelligence) models in real-time. The computational demands of modern machine learning algorithms for image analysis, object detection, and segmentation typically exceed the processing resources available in surgical display hardware. Consequently, AI-enhanced surgical guidance cannot be implemented directly on existing surgical devices without substantial hardware modifications.

Another challenge involves the diversity of surgical device ecosystems. Different surgical devices from various manufacturers may employ distinct data formats, communication protocols, and visualization software architectures. For example, a vitrectomy probe may accept only simple bounding box coordinates for overlay visualization, while a phacoemulsification system may support more sophisticated augmentation including text annotations and six-degree-of-freedom coordinate data. As a result, integrating AI-generated information into devices would result in device-specific software modifications and increased costs to maintain the separate software systems.

A further challenge relates to the real-time performance requirements of surgical applications. Surgical procedures demand low-latency visualization where augmented information appears synchronized with live video streams. Excessive data transmission rates may overwhelm device processing capabilities, causing frame rate degradation, memory overflow, or rendering delays. Conversely, insufficient update rates may result in outdated or misleading augmentation.

In some embodiments, the methods and system described in this disclosure may assist in addressing some or all of these challenges. The methods and system described herein present a surgical system architecture that integrates a local computing device with multiple surgical devices via a communication interface.

The local computing device may host AI models and may perform computationally intensive inference operations on surgical video streams to generate overlay visualizations for the surgical video streams. The overlay visualizations may augment the surgical video stream by providing real-time visual guidance and/or contextual information that may be superimposed directly onto the surgical field view. For example, bounding boxes may be rendered around anatomical structures, segmentation masks may be overlaid to delineate boundaries, depth indicators may be displayed to convey spatial relationships, text annotations may appear adjacent to identified structures, and/or alert indicators may be presented, among other data. The augmented video stream may thereby transform a standard surgical view into a display that may reduce cognitive load on the surgeon, improve anatomical identification accuracy, decrease procedural time, and/or enhance overall surgical outcomes without the surgeon having to divert attention from the surgical field.

In some embodiments, the communication interface may function as an intermediary between the local computing device and the surgical devices. In these and other embodiments, the communication interface may obtain the surgical video streams from the medical devices and provide the surgical video streams to the local computing device. The communication interface may also obtain presentation data, e.g., the overlay visualizations, generated by the local computing device. The communication interface may map the presentation data to data structures accepted by the individual medical devices. In these and other embodiments, each data structure may direct the corresponding medical device how to render the presentation data. The communication interface may also direct the formatted data structures to the medical devices for processing and visualization, thereby allowing for the presentation data to be overlaid on the surgical video streams.

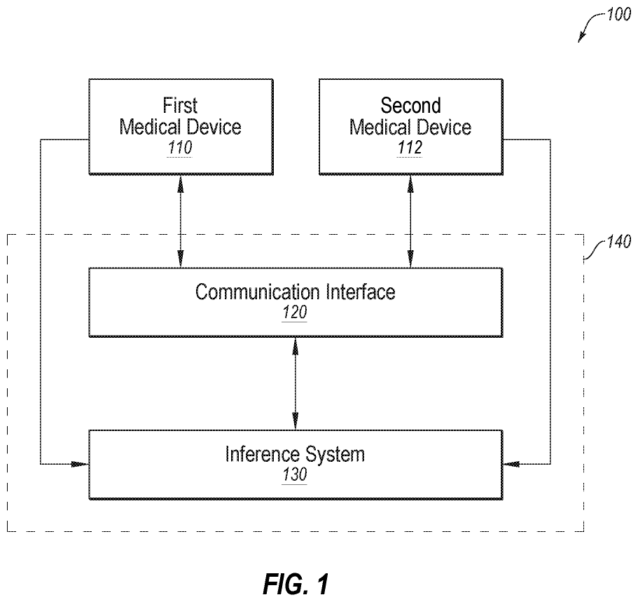

Turning to the figures, FIG. 1 illustrates a block diagram of an environment 100 for interfacing an inference system on a local computing device with medical devices. The environment 100 may be arranged in accordance with at least one embodiment described in the present disclosure. The environment 100 may include a first medical device 110, a second medical device 112, a communication interface 120, and an inference system 130.

In some embodiments, the first medical device 110 may be any type of imaging device configured to generate image data during a surgical procedure. In these and other embodiments, the image data may include video streams captured by imaging components integrated within the first medical device 110. For example, the first medical device 110 may be configured to capture video of a surgical field at frame rates suitable for real-time visualization. In some embodiments, the first medical device 110 may include or may be connected to a device with display capabilities for presenting image data to a surgeon or other medical personnel during a surgical procedure.

As an example, the first medical device 110 may be an ophthalmic medical device, such as a vitrectomy probe, a phacoemulsification probe, an illumination probe, or some other ophthalmic medical device. Alternately or additionally, the first medical device 110 may be a biopsy probe, an ablation probe, an electrocautery probe, and/or a neurostimulation probe among other types of medical devices that generate image data.

In some embodiments, the first medical device 110 may include a rendering engine configured to generate and display visual information. The rendering engine may be a software component residing within the first medical device 110 that processes graphical data and produces visual output for presentation to a surgeon or other medical personnel during a surgical procedure. In these and other embodiments, the rendering engine may be configured to render overlays on the image data generated by the first medical device 110. For example, the first medical device 110 may generate overlays representing device operational parameters such as aspiration pressure readings, infusion flow rates, vacuum levels, and/or cutting speeds, among other operational parameters. Alternately or additionally, the rendering engine may generate overlays displaying intraocular coherence (IOC) values, phacoemulsification power settings, vitrectomy probe status indicators, and/or illumination intensity levels, among other values. In these and other embodiments, the overlays generated by the rendering engine of the first medical device 110 may not require external processing or artificial intelligence inference to generate. The rendering engine may be configured to continuously update the overlays in real-time as operational parameters change during the surgical procedure.

In some embodiments, in addition to displaying the image data, the first medical device 110 may be configured to transmit the image data to the communication interface 120. Responsive to providing the image data to the communication interface 120, the first medical device 110 may obtain presentation data from the communication interface 120. The presentation data may include overlay visualizations generated by the inference system 130 based on the image data provided by the first medical device 110 to the communication interface 120. The first medical device 110 may be configured to render the presentation data along with the image data and/or other visual information generated by the first medical device 110. The rendering may occur in real-time such that the overlay visualizations appear synchronized with a video stream of a surgical field.

In some embodiments, the first medical device 110 may be configured to render the presentation data along with the image data and/or other visual information generated by the first medical device 110 without requiring software modifications.

In some embodiments, the first medical device 110 may be configured to accept the presentation data in a data structure format. In some embodiments, the data structure may be specific to the first medical device 110 and may represent a format for data that may be provided to the first medical device 110 for proper rendering of the data by the first medical device 110. For example, a first medical device 110 may accept data structures comprising simple bounding box coordinates formatted as four-value tuples specifying x-position, y-position, width, and height parameters, while another medical device may accept a more complex data structure. In these and other embodiments, the data structure may conform to a rendering application programming interface (API) of the first medical device 110. In some embodiments, the data structure may reference predefined graphical objects stored in a rendering library of the first medical device 110. In these and other embodiments, the data structure may include object identifiers and transformation parameters such as position coordinates, scale factors, and rotation angles that the first medical device 110 may apply to the predefined objects during rendering.

In some embodiments, the first medical device 110 may transmit information to the communication interface 120 regarding the data structure format that the first medical device 110 may accept for rendering presentation data. In these and other embodiments, the first medical device 110 may generate and send a capability profile message to the communication interface 120 upon initial connection or in response to a query from the communication interface 120. The capability profile message may include metadata specifying the data structure format, including field definitions, data types, coordinate systems, and rendering constraints that the first medical device 110 may support.

In some embodiments, the first medical device 110 may be configured to generate device status information. The device status information may include metrics related to the operational state of the first medical device 110, such as resource usage data. For example, the resource usage data may include memory usage statistics indicating the amount of random access memory currently consumed by rendering operations; processing latency measurements indicating the time delay between receiving presentation data and completing the rendering of overlay visualizations, among other statistics indicating a capability of the first medical device 110 to render overlays on the image data.

In some embodiments, the first medical device 110 may be configured to transmit the device status information to the inference system 130. In some embodiments, the transmission of the device status information may occur via the communication interface 120 or via a direct communication pathway that bypasses the communication interface 120. In these and other embodiments, the direct communication pathway may be a unidirectional data channel configured for transmitting the device status information without transmitting the image data.

In some embodiments, the second medical device 112 may be the same or a different type of medical device than the first medical device 110. In these and other embodiments, the second medical device 112 may be configured similarly to the first medical device 110. For example, the second medical device 112 may be configured to generate image data during a surgical procedure, transmit the image data to the communication interface 120, and receive presentation data from the communication interface 120. In these and other embodiments, the second medical device 112 may be configured to render the presentation data on a display screen. In some embodiments, the second medical device 112 may be configured to accept presentation data in data structure with a format that is the same or different from the format accepted by the first medical device 110.

In some embodiments, the communication interface 120 and the first medical device 110 and from the second medical device 112 may exchange data. For example, the communication interface 120 may be configured to obtain image data from the first medical device 110 and from the second medical device 112. The communication interface 120 may be further configured to provide the image data to the inference system 130 for processing and obtain presentation data from the inference system 130 for sending to the first and second medical devices 110 and 112. As such, the communication interface 120 may be configured to function as an intermediary between the inference system 130 and the first and second medical devices 110 and 112.

In some embodiments, the communication interface 120 and the first medical device 110 and from the second medical device 112 may exchange data via network connections (wired or wireless), serial interfaces, or USB interfaces. In these and other embodiments, the data may be serialized according to device-specific protocols, such as JSON for text-based data interchange, binary serialization for performance-critical applications, or custom protocols defined by device manufacturers.

In some embodiments, the communication interface 120 may include a software communication layer that facilitates data transmission between the inference system 130 and the first and second medical devices 110 and 112. The software communication layer may be implemented as a message broker that routes data between system components. For example, the communication interface 120 may utilize ZeroMQ as the message broker. A message structure of the data may be standardized JSON and may contain a device ID, data payload, data type, and a timestamp. The device ID (Identification) may identify one of the first and second medical devices 110 and 112. The data payload may be the data to be displayed or utilized by one of the first and second medical devices 110 and 112 (e.g., bounding box coordinates, segmentation masks, guidance cues). The data type may be specific to the type of data being transmitted (e.g., image, coordinates, text). The timestamp may be used for synchronization and data integrity.

In some embodiments, the communication interface 120 may be configured to query medical devices to obtain capability information. In these and other embodiments, the communication interface 120 may be configured to detect when a new medical device connects to the environment 100. The communication interface 120 may be configured to initiate a handshake protocol with the newly connected medical device. The communication interface 120 may be configured to transmit a capability query message to the newly connected medical device. The capability query message may request information about the data structure formats accepted by the medical device. The communication interface 120 may be configured to receive a capability response message from the medical device. The capability response message may include a capability profile specifying the supported data types and formats. The communication interface 120 may be configured to store the capability profile in a device registry. The communication interface 120 may be configured to use the capability profile to format presentation data for the medical device without requiring manual configuration.

In some embodiments, the communication interface 120 may be configured to maintain a device registry. The device registry may store information about each medical device connected to the environment 100. The device registry may include device identification information for the first medical device 110 and the second medical device 112. The device identification information may include unique device identifiers assigned to each medical device. Alternately or additionally, the device registry may store the capability profiles for each medical device.

In some embodiments, the communication interface 120 may be configured to format the presentation data obtained from the inference system 130 before providing the presentation data to the first medical device 110 and the second medical device 112. In these and other embodiments, the communication interface 120 may be configured to receive presentation data in a standardized format from the inference system 130 and map the presentation data to a data structure associated with the medical device designated for the presentation data.

In some embodiments, the communication interface 120 may be configured to map the presentation data to the data structure for the appropriate medical device by retrieving the device capability profile from the device registry and selecting a corresponding format adapter module. The format adapter module may be software configured to parse the presentation data received from the inference system 130 and extract relevant components such as bounding box coordinates, segmentation masks, text annotations, or depth information. The format adapter module may transform the extracted components into the data structure format specified by the target medical device, which may include converting coordinate systems, scaling values to match device resolution, serializing data according to device protocol requirements, or mapping data elements to predefined graphical objects, among other functions.

In some embodiments, the mapping process may occur independently for each connected medical device, allowing the communication interface 120 to generate different data structure formats when multiple medical devices with different capabilities are connected to the communication interface 120. The communication interface 120 may perform the mapping operation in real-time as presentation data is received from the inference system 130, maintaining synchronization between the AI-generated augmentation information and the video streams displayed by the connected medical devices.

In some embodiments, the communication interface 120 may validate the formatted data structure against device specifications to ensure compliance with expected data types, value ranges, and structural requirements before transmission.

In some embodiments, the communication interface 120 may be configured to synchronize presentation data with video frames displayed on the first medical device 110 and the second medical device 112. To synchronize presentation data, the communication interface 120 may be configured to include timestamp information with the presentation data. The timestamp information may indicate the time at which the presentation data was generated by the inference system 130. The first medical device 110 and the second medical device 112 may be configured to use the timestamp information to align overlay visualizations with corresponding video frames.

In some embodiments, the communication interface 120 may be configured to transmit presentation data at a frequency that matches or exceeds the video frame rate of the medical devices. For example, if the medical devices display video at thirty frames per second, the communication interface 120 may be configured to transmit presentation data at a rate of at least thirty updates per second. In some embodiments, the communication interface 120 may be configured to transmit presentation data at a lower frequency than the video frame rate. The lower frequency may be sufficient for overlay visualizations that do not require frame-by-frame updates. The communication interface 120 may be configured to determine the appropriate transmission frequency based on the type of overlay visualization and the performance characteristics of the first medical device 110 and the second medical device 112.

In some embodiments, the inference system 130 may be configured to execute artificial intelligence (AI) models for analyzing image data. The inference system 130 may be configured to receive image data from the communication interface 120. The image data may include image data, e.g., videos streams captured by the first medical device 110 and the second medical device 112. The inference system 130 may be configured to process the image data using trained AI models.

In some embodiments, the AI models may include object detection models configured to identify anatomical structures in the image data. Alternately or additionally, the AI models may include segmentation models configured to delineate boundaries of anatomical structures at a pixel level, classification models configured to categorize surgical phases or tissue types, and/or depth estimation models configured to compute spatial relationships between structures in the surgical field.

In some embodiments, the inference system 130 may be configured to generate presentation data based on the output of the AI models. The presentation data may include bounding box coordinates that define rectangular regions around identified anatomical structures, segmentation masks that assign class labels to individual pixels in the image data, text annotations that provide labels or confidence scores for identified structures, depth indicators that convey three-dimensional spatial relationships, alert indicators that flag procedural irregularities or safety concerns detected by the AI models, among other data. The inference system 130 may be configured to format the presentation data in a standardized format. The standardized format may include structured data objects with fields for different types of overlay information. The standardized format may include device identification information indicating the target medical device for the presentation data. Alternately or additionally, the standardized format may include timestamp information indicating when the presentation data was generated.

In some embodiments, the inference system 130 may be configured to transmit the presentation data to the communication interface 120. The transmission may occur via a high-bandwidth network connection. For example, the network connection may be an Ethernet connection. The network connection may provide low-latency data transfer suitable for real-time surgical applications. The inference system 130 may be configured to transmit the presentation data continuously as new image data is processed. The inference system 130 may be configured to transmit presentation data at a rate that matches the video frame rate of the medical devices. For example, if the medical devices display video at thirty frames per second, the inference system 130 may be configured to generate and transmit presentation data at a rate of thirty updates per second.

In some embodiments, the inference system 130 may be configured to receive device status information from the medical devices. The device status information may be transmitted via a direct communication pathway that bypasses the communication interface 120. The direct communication pathway may be a unidirectional data channel configured for transmitting device health telemetry.

In some embodiments, the inference system 130 may be configured to analyze the device status information to detect performance degradation of the medical devices. The device status information may include frame rate measurements, memory usage statistics, and processing latency measurements, among other information. As an example, performance degradation may be indicated by a decrease in frame rate below a threshold value. Alternately or additionally, performance degradation may be indicated by an increase in memory usage above a threshold percentage and/or by an increase in processing latency above a threshold time duration.

In response to performance degradation, the inference system 130 may be configured to adjust the processing of image data based on the device status information. For example, if device status information indicates that a medical device is experiencing high memory usage, the inference system 130 may be configured to reduce the complexity of the AI models or reduce the frequency of inference operations.

In some embodiments, the communication interface 120 may be configured to implement adaptive throttling based on the performance degradation. The adaptive throttling may adjust the transmission frequency of the presentation data to the medical devices. For example, if the frame rate decreases by ten percent, the transmission frequency may be reduced by ten percent. The adaptive throttling may implement a threshold-based reduction where the transmission frequency is reduced by a fixed amount when performance degradation exceeds a threshold.

In some embodiments, the inference system 130 may be configured to execute multiple artificial intelligence models concurrently. The multiple models may process image data from different medical devices simultaneously. Alternately or additionally, the multiple models may perform different types of analysis on the same image data. For example, a first model may perform object detection to identify anatomical structures, while a second model may perform segmentation to delineate structure boundaries. The inference system 130 may be configured to combine the outputs of multiple models to generate comprehensive presentation data for a single medical device.

In some embodiments, a system 140 may encompass the communication interface 120 and the inference system 130. The system 140 may represent a local computing device that hosts both the communication interface software and the inference system hardware and software. The system 140 may be configured to operate as an integrated unit that performs artificial intelligence inference and manages communication with medical devices. The system 140 may be positioned in proximity to the medical devices such that the system 140 may be configured to operate independently without external network connectivity. Furthermore, the system 140 may be configured to maintain low-latency operation suitable for real-time surgical applications, such as a dedicated edge computing device.

In some embodiments, the environment 100 may be configured to support multiple different types of medical procedures. For example, the medical procedures may include ophthalmic surgeries such as vitreoretinal surgery, cataract surgery, glaucoma surgery, and laser eye surgery, among other types of medical procedures. The medical procedures may include other types of procedures where real-time visual augmentation may be useful.

Modifications, additions, or omissions may be made to the environment 100 without departing from the scope of the present disclosure. For example, in some embodiments, the communication interface 120 and the inference system 130 may be implemented as separate physical devices connected via a network. In some embodiments, the environment 100 may include additional components such as data storage systems for logging surgical data, user interface devices for configuring system parameters, or monitoring displays for observing system performance. In some embodiments, the direct communication pathway between the medical devices and the inference system 130 may be omitted, and device status information may be transmitted via the communication interface 120.

As another example, the environment 100 may include additional medical devices beyond the first medical device 110 and the second medical device 112. The additional medical devices may be configured similarly to the first and second medical devices. The additional medical devices may be configured to generate image data and transmit the image data to the communication interface 120. The additional medical devices may be configured to receive presentation data from the communication interface 120 and render overlay visualizations. The communication interface 120 may be configured to manage communication with any number of medical devices. The communication interface 120 may be configured to maintain device registry entries for each connected medical device and to format presentation data for each medical device based on each medical device's capability profile.

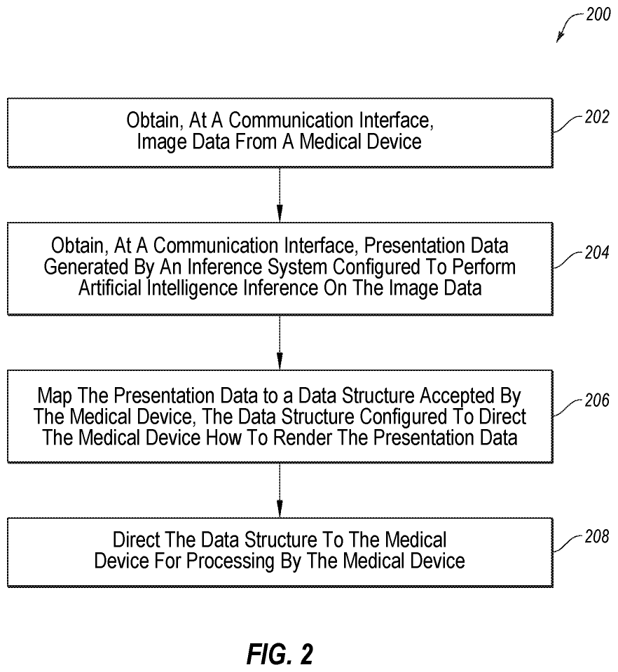

FIG. 2 illustrates a flowchart of an example method 200 of interfacing with medical devices, in accordance with one or more embodiments of the present disclosure. The method 200 may be performed by any suitable system, apparatus, or device. For example, the method 200 may be implemented by the communication interface 120 described throughout the present disclosure. Although illustrated with discrete blocks, the steps and operations associated with one or more blocks of the method 200 may be divided into additional blocks, combined into fewer blocks, or eliminated, depending on the particular implementation.

The method 200 may include blocks 202, 204, 206, and 208. At block 202, image data may be obtained at a communication interface from a medical device. The image data may be captured by an imaging component integrated within the medical device. The image data may include a video stream of a surgical field during a medical procedure that involves the medical device.

At block 204, presentation data may be obtained at the communication interface. The presentation data may be generated by an inference system. The inference system may be configured to perform AI inference on the image data where the image data is obtained by the inference system via the communication interface. In some embodiments, the inference system may execute one or more trained AI models that may process the image data to generate the presentation data. In these and other embodiments, the presentation data may include overlay visualizations.

In some embodiments, the inference system may format the presentation data in a standardized format. The standardized format may include structured data objects with fields for different types of overlay information.

At block 206, the presentation data may be mapped to a data structure accepted by the medical device. The data structure may be configured to direct the medical device how to render the presentation data. In some embodiments, the communication interface may retrieve a device capability profile from a device registry. The device capability profile may specify the data structure format accepted by the medical device. In some embodiments, the communication interface and the inference system may be part of the same system. The same system may be a local computing device. The local computing device may host both the communication interface software and the inference system hardware and software. The local computing device may be positioned in proximity to the medical device. The local computing device may operate independently without external network connectivity. The local computing device may maintain low-latency operation suitable for real-time surgical applications.

At block 208, the data structure may be directed to the medical device for processing by the medical device. In some embodiments, processing of the data structure may include generating an overlay for video data presented by the medical device based on the data structure. In some embodiments, the image data obtained from the medical device may be included in video data presented by the medical device. The video data may be displayed on a display screen of the medical device. In these and other embodiments, the data structure may be rendered by the medical device as an overlay for the video data. The overlay may be superimposed on the video data. The overlay may include visual elements that augment the live view of the surgical field. In some embodiments, the medical device may render the overlay using a rendering engine. The rendering engine may be a software component residing within the medical device.

One skilled in the art will appreciate that, for this and other processes, operations, and methods disclosed herein, the functions and/or operations performed may be implemented in differing order. Furthermore, the outlined functions and operations are only provided as examples, and some of the functions and operations may be optional, combined into fewer functions and operations, or expanded into additional functions and operations without detracting from the essence of the disclosed embodiments.

For example, in some embodiments, resource usage data of the medical device may be obtained from the medical device. The resource usage data may include memory usage statistics. The memory usage statistics may indicate the amount of random-access memory currently consumed by rendering operations. In these and other embodiments, the resource usage data may be transmitted from the medical device to the inference system. The transmission may occur via a direct communication pathway that may bypass the communication interface.

In some embodiments, a timing for directing the data structure to the medical device may be adjusted based on the resource usage data. The new transmission frequency may be lower than a current transmission frequency. In some embodiments, adjusting the timing for directing the data structure to the medical device based on the resource usage data may include delaying sending the data structure to the medical device in response to the resource usage data indicating resource usage of the medical device satisfying a threshold. The threshold may be a memory usage threshold.

As another example, in some embodiments, identity data of the medical device may be obtained before obtaining the image data from the medical device. The identity data may include a unique device identifier. The identity data may be transmitted from the medical device to the communication interface. The transmission may occur during an initial connection or in response to a query from the communication interface. The data structure to which the presentation is mapped may be selected using the identity data.

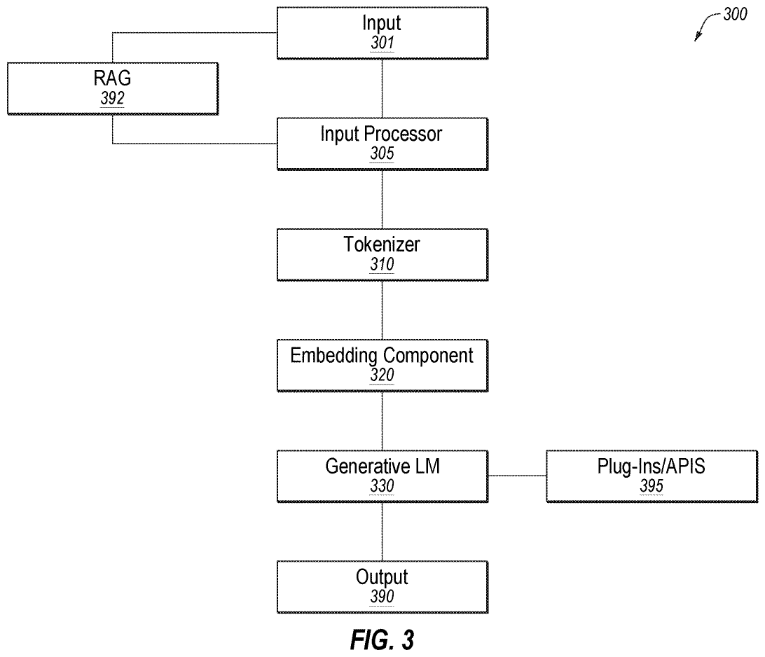

FIG. 3 is a block diagram of an example generative language model system 300 suitable for use in implementing at least some embodiments of the present disclosure. In the example illustrated in FIG. 3, the generative language model system 300 includes a retrieval augmented generation (RAG) component 392, an input processor 305, a tokenizer 310, an embedding component 320, plug-ins/APIs 395, and a generative language model (LM) 330 (which may include an LLM (large language model), a VLM (vision language model), a MMLM (multi-modal language model), a VLA (vision language action) model, etc.). The generative language model system 300 may be an example of a system that operates on the inference system 130.

At a high level, the input processor 305 may receive an input 301 comprising text and/or other types of input data (e.g., audio data, video data, image data, sensor data), depending on the architecture of the generative LM 330 (e.g., LLM/LRM/VLM/MMLM/etc.). Taking raw input text as an example, the input processor 305 may prepare raw input text in various ways. For example, the input processor 305 may perform various types of text filtering to remove noise (e.g., special characters, punctuation, HTML (Hypertext Markup Language) tags, stopwords, portions of an image(s), portions of audio, etc.) from relevant textual content.

In some embodiments, a RAG component 392 (which may include one or more RAG models, and/or may be performed using the generative LM 330 itself) may be used to retrieve additional information to be used as part of the input 301 or prompt. RAG may be used to enhance the input to the LLM/LRM/VLM/MMLM/etc. with external knowledge, so that answers to specific questions or queries or requests are more relevant—such as in a case where specific knowledge is required. The RAG component 392 may fetch this additional information (e.g., grounding information, such as grounding text/image/video/audio/USD/CAD/etc.) from one or more external sources, which can then be fed to the LLM/LRM/VLM/MMLM/etc. along with the prompt to improve accuracy of the responses or outputs of the model.

For example, in some embodiments, the input 301 may be generated using the query or input to the model (e.g., a question, a request, etc.) in addition to data retrieved using the RAG component 392. In some embodiments, the input processor 305 may analyze the input 301 and communicate with the RAG component 392 (or the RAG component 392 may be part of the input processor 305, in embodiments) in order to identify relevant text and/or other data to provide to the generative LM 330. The RAG component 392 may use various RAG techniques such as, naïve RAG, modular RAG techniques, and/or Graph RAG.

The tokenizer 310 may segment the (e.g., processed) text data into smaller units (tokens) for subsequent analysis and processing. The tokens may represent individual words, subwords, characters, portions of audio/video/image/etc., depending on the implementation. For example, the tokenizer 310 may convert the (e.g., processed) text into a structured format according to tokenization schema being implemented in the particular embodiment.

The embedding component 320 may use any known embedding technique to transform discrete tokens into (e.g., dense, continuous vector) representations of semantic meaning. For example, the embedding component 320 may use pre-trained word embeddings (e.g., Word2Vec, GloVe, or FastText), one-hot encoding, Term Frequency-Inverse Document Frequency (TF-IDF) encoding, one or more embedding layers of a neural network, and/or otherwise. In some implementations in which the input 301 includes video data, the input processor 301 may extract frames or apply resizing to extracted frames, and the embedding component 320 may extract features such as optical flow embeddings or video embeddings and/or may encode temporal information or sequences of frames.

The generative LM 330 and/or other components of the generative LM system 300 may use different types of neural network architectures depending on the implementation. For example, transformer-based architectures such as those used in models like GPT (Generative Pre-trained Transformer) may be implemented, and may include self-attention mechanisms. Some other non-limiting example architectures include transformers (e.g., encoder-decoder, decoder only, multi-modal), RNNs (Recurrent Neural Network), LSTMs (Long Short-Term Memory), fusion models, diffusion models, cross-modal embedding models that learn joint embedding spaces, graph neural networks (GNNs), hybrid architectures combining different types of architectures adversarial networks like generative adversarial networks or GANs or adversarial autoencoders (AAEs) for joint distribution learning, linear-time sequence modeling with selective state space modeling (SSM) architectures (e.g., Mamba LLM architectures), and/or others. As such, depending on the implementation and architecture, the embedding component 320 may apply an encoded representation of the input 301 to the generative LM 330, and the generative LM 330 may process the encoded representation of the input 301 to generate an output 390, which may include responsive text and/or other types of data.

As described herein, in some embodiments, the generative LM 330 may be configured to access or use—or capable of accessing or using—plug-ins/APIs 395 (which may include one or more plug-ins, application programming interfaces (APIs), databases, data stores, repositories, etc.). These and other architectures for LLMs/VLMs/MMLMs/VLAs/etc. described herein are meant simply as examples, and other suitable architectures may be implemented within the scope of the present disclosure.

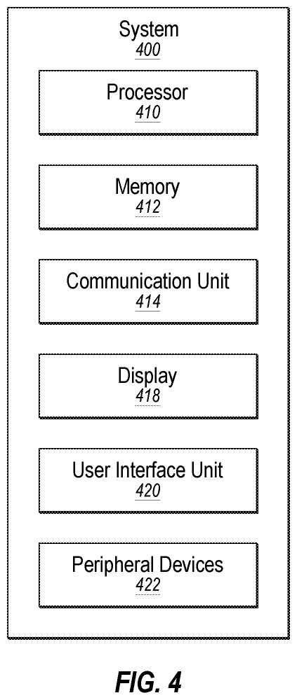

FIG. 4 illustrates an example system 400 that may be used during transcription of communications. The system 400 may be arranged in accordance with at least one embodiment described in the present disclosure. The system 400 may include a processor 410, memory 412, a communication unit 414, a display 418, a user interface unit 420, and a peripheral device 422, which all may be communicatively coupled. In some embodiments, the system 400 may be part of any of the systems or devices described in this disclosure.

Generally, the processor 410 may include any suitable special-purpose or general-purpose computer, computing entity, or processing device including various computer hardware or software modules and may be configured to execute instructions stored on any applicable computer-readable storage media. For example, the processor 410 may include a microprocessor, a microcontroller, a parallel processor such as a graphics processing unit (GPU) or tensor processing unit (TPU), a digital signal processor (DSP), an application-specific integrated circuit (ASIC), a Field-Programmable Gate Array (FPGA), quantum computer, or any other digital or analog circuitry configured to interpret and/or to execute program instructions and/or to process data.

Although illustrated as a single processor in FIG. 4, it is understood that the processor 410 may include any number of processors distributed across any number of networks or physical locations that are configured to perform individually or collectively any number of operations described herein. In some embodiments, the processor 410 may interpret and/or execute program instructions and/or process data stored in the memory 412. In some embodiments, the processor 410 may execute the program instructions stored in the memory 412.

For example, in some embodiments, the processor 410 may execute program instructions stored in the memory 412 that are related to selecting a transcription generation technique for a communication session such that the system 400 may perform or direct the performance of the operations associated therewith as directed by the instructions. In these and other embodiments, the instructions may be used to perform one or more operations of the methods described in this disclosure.

The memory 412 may include computer-readable storage media or one or more computer-readable storage mediums for carrying or having computer-executable instructions or data structures stored thereon. Such computer-readable storage media may be any available media that may be accessed by a general-purpose or special-purpose computer, such as the processor 410.

By way of example, and not limitation, such computer-readable storage media may include non-transitory computer-readable storage media including Random Access Memory (RAM), Read-Only Memory (ROM), Electrically Erasable Programmable Read-Only Memory (EEPROM), Compact Disc Read-Only Memory (CD-ROM) or other optical disk storage, magnetic disk storage or other magnetic storage devices, flash memory devices (e.g., solid state memory devices), or any other storage medium which may be used to carry or store particular program code in the form of computer-executable instructions or data structures and which may be accessed by a general-purpose or special-purpose computer. Combinations of the above may also be included within the scope of computer-readable storage media.

Computer-executable instructions may include, for example, instructions and data configured to cause the processor 410 to perform a certain operation or group of operations as described in this disclosure. In these and other embodiments, the term “non-transitory” as explained in the present disclosure should be construed to exclude only those types of transitory media that were found to fall outside the scope of patentable subject matter in the Federal Circuit decision of In re Nuijten, 500 F.3d 1346 (Fed. Cir. 2007). Combinations of the above may also be included within the scope of computer-readable media.

The communication unit 414 may include any component, device, system, or combination thereof that is configured to transmit or receive information over a network. In some embodiments, the communication unit 414 may communicate with other devices at other locations, the same location, or even other components within the same system. For example, the communication unit 414 may include a modem, a network card (wireless or wired), an infrared communication device, a wireless communication device (such as an antenna), and/or chipset (such as a Bluetooth device, an IEEE 802.6 device (e.g., Metropolitan Area Network (MAN)), a WiFi device, a WiMax device, cellular communication facilities, etc.), and/or the like. The communication unit 414 may permit data to be exchanged with a network and/or any other devices or systems described in the present disclosure.

The display 418 may be configured as one or more displays, like an (liquid crystal display), LED (light emitting diode), OLED (organic light emitting diode), Braille terminal, or other type of display. The display 418 may be configured to present video, text captions, user interfaces, and other data as directed by the processor 410. The user interface unit 420 may include any device to allow a user to interface with the system 400. For example, the user interface unit 420 may include a mouse, a track pad, a keyboard, buttons, camera, and/or a touchscreen, among other devices. The user interface unit 420 may receive input from a user and provide the input to the processor 410. In some embodiments, the user interface unit 420 and the display 418 may be combined.

The peripheral devices 422 may include one or more devices. For example, the peripheral devices may include a microphone, an imager, and/or a speaker, among other peripheral devices. Modifications, additions, or omissions may be made to the system 400 without departing from the scope of the present disclosure. For example, in some embodiments, the system 400 may include any number of other components that may not be explicitly illustrated or described. Further, depending on certain implementations, the system 400 may not include one or more of the components illustrated and described.

As indicated above, the embodiments described herein may include the use of a special purpose or general-purpose computer (e.g., the processor 410 of FIG. 4) including various computer hardware or software modules, as discussed in greater detail below. Further, as indicated above, embodiments described herein may be implemented using computer-readable media (e.g., the memory 412 of FIG. 4) for carrying or having computer-executable instructions or data structures stored thereon.

Generally speaking, network environments suitable for use in implementing embodiments of the disclosure may include one or more client devices, servers, network attached storage (NAS), other backend devices, and/or other device types. The client devices, servers, and/or other device types (e.g., each device) may be implemented on one or more instances of the computing systems(s) 400 of FIG. 4—e.g., each device may include similar components, features, and/or functionality of the computing systems(s) 400. In addition, where backend devices (e.g., servers, NAS, etc.) are implemented, the backend devices may be included as part of a data center (such as, but not limited to, those described herein).

Components of a network environment may communicate with each other via a network(s), which may be wired, wireless, or both. The network may include multiple networks, or a network of networks. Compatible network environments may include one or more peer-to-peer network environments—in which case a server may not be included in a network environment—and one or more client-server network environments—in which case one or more servers may be included in a network environment. In peer-to-peer network environments, functionality described herein with respect to a server(s) may be implemented on any number of client devices.

In at least one embodiment, a network environment may include one or more cloud-based network environments, a distributed computing environment, a combination thereof, etc. A cloud-based network environment may be private (e.g., limited to a single organization), may be public (e.g., available to many organizations), and/or a combination thereof (e.g., a hybrid cloud environment).

The various features illustrated in the drawings may be, but are not necessarily, drawn to scale. The illustrations presented in the present disclosure are not meant to be actual views of any particular apparatus (e.g., device, system, etc.) or method, but are merely idealized representations that are employed to describe various embodiments of the disclosure. Accordingly, the dimensions of the various features may be arbitrarily expanded or reduced for clarity. In addition, some of the drawings may be simplified for clarity. Thus, the drawings may not depict all of the components of a given apparatus (e.g., device) or all operations of a particular method.

Terms used in the present disclosure and especially in the appended claims (e.g., bodies of the appended claims) are generally intended as “open” terms (e.g., the term “including” should be interpreted as “including, but not limited to,” the term “having” should be interpreted as “having at least,” the term “includes” should be interpreted as “includes, but is not limited to,” among others).

Relative terms used in the present disclosure and especially in the appended claims (e.g., bodies of the appended claims) are generally intended as falling within manufacturing tolerances and/or within scope reasonably understood by a person of skill in the art. For example, if two components are identified as being the “same” size, there may be variations consistent with manufacturing variances. Terms describing “approximately,” “similar,” “substantially,” or other terms designating similarity may convey within ten percent of the comparative value. For example, two components that are approximately the same size would be understood to be of a size within ten percent of each other.

Additionally, if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations.

In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” or “one or more of A, B, and C, etc.” is used, in general such a construction is intended to include A alone, B alone, C alone, A and B together, A and C together, B and C together, or A, B, and C together, etc.

Further, any disjunctive word or phrase presenting two or more alternative terms, whether in the description, claims, or drawings, should be understood to contemplate the possibilities of including one of the terms, either of the terms, or both terms. For example, the phrase “A or B” should be understood to include the possibilities of “A” or “B” or “A and B.” However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations.

Additionally, the use of the terms “first,” “second,” “third,” etc., are not necessarily used herein to connote a specific order or number of elements. Generally, the terms “first,” “second,” “third,” etc., are used to distinguish between different elements as generic identifiers. Absence a showing that the terms “first,” “second,” “third,” etc., connote a specific order, these terms should not be understood to connote a specific order. Furthermore, absence a showing that the terms “first,” “second,” “third,” etc., connote a specific number of elements, these terms should not be understood to connote a specific number of elements. For example, a first widget may be described as having a first side and a second widget may be described as having a second side. The use of the term “second side” with respect to the second widget may be to distinguish such side of the second widget from the “first side” of the first widget and not to connote that the second widget has two sides.

All examples and conditional language recited in the present disclosure are intended for pedagogical objects to aid the reader in understanding the invention and the concepts contributed by the inventor to furthering the art, and are to be construed as being without limitation to such specifically recited examples and conditions. Although embodiments of the present disclosure have been described in detail, it should be understood that the various changes, substitutions, and alterations could be made hereto without departing from the spirit and scope of the present disclosure.

What is claimed is:

1. A method of interfacing with medical devices, the method comprising:
obtaining, at a communication interface, image data from a medical device;
obtaining, at the communication interface, presentation data generated by an inference system configured to perform artificial-intelligence inference on the image data;
mapping the presentation data to a data structure accepted by the medical device, the data structure configured to direct the medical device how to render the presentation data; and
directing the data structure to the medical device for processing by the medical device.

2. The method of claim 1, wherein the communication interface and the inference system are part of the same system.

3. The method of claim 2, further comprising:
obtaining, from the medical device, a resource usage data of the medical device; and
adjusting a timing for directing the data structure to the medical device based on the resource usage data.

4. The method of claim 3, wherein adjusting the timing for directing the data structure to the medical device based on the resource usage data includes, in response to the resource usage data indicating resource usage of the medical device satisfying a threshold, delaying sending the data structure to the medical device.

5. The method of claim 1, wherein the image data is included in video data presented by the medical device and the data structure is rendered by the medical device as an overlay for the video data.

6. The method of claim 1, further comprising:
obtaining, at the communication interface, second image data from a second medical device;
obtaining, at the communication interface, second presentation data generated by the inference system using the second image data; and
mapping the second presentation data to a second data structure accepted by the second medical device, the second data structure configured to direct the second medical device how to render the second presentation data.

7. The method of claim 1, further comprising:
before obtaining the image data from the medical device, obtaining identity data of the medical device; and
selecting the data structure using the identity data.

8. The method of claim 1, further comprising validating the data structure for compliance with the medical device, before directing the data structure to the medical device.

9. A system for interfacing with medical devices, the system comprising:
an inference system configured to perform artificial-intelligence inference on data;
a communication interface configured to:
obtain image data from a medical device;
obtain presentation data generated by the inference system using the image data;
map the presentation data to a data structure accepted by the medical device, the data structure configured to direct the medical device how to render the presentation data; and
direct the data structure to the medical device for processing by the medical device.

10. The system of claim 9, wherein the image data is included in video data presented by the medical device and the data structure is rendered by the medical device as an overlay for the video data.

11. The system of claim 9, wherein the inference system is configured to:
obtain from the medical device, a resource usage data of the medical device; and
adjust a timing for directing the data structure to the medical device based on the resource usage data.

12. The system of claim 11, wherein adjusting the timing for directing the data structure to the medical device based on the resource usage data includes, in response to the resource usage data indicating resource usage of the medical device satisfying a threshold, delaying sending the data structure to the medical device.

13. The system of claim 9, wherein the communication interface is further configured to:
obtain second image data from a second medical device;
obtain second presentation data generated by the inference system using the second image data; and
map the second presentation data to a second data structure accepted by the second medical device, the second data structure configured to direct the second medical device how to render the second presentation data.

14. The system of claim 9, wherein the communication interface is further configured to:
before obtaining the image data from the medical device, obtain identity data of the medical device; and
select the data structure using the identity data.

15. The system of claim 9, wherein the communication interface is further configured to validate the data structure for compliance with the medical device, before directing the data structure to the medical device.
